(19) United States
(12) Patent Application Publication
(10) Pub. No.: US 2015/0314095 A1
(43) Pub. Date: Nov. 5, 2015

(54) PATIENT INTERFACE CUSHION WITH INTEGRAL STRAPS

(71) Applicant: Koninklijke Philips N.V., Eindhoven (NL)

(72) Inventors: Kevin Daniel HIMES, JR., Irwin, PA (US); Elizabeth EURY, Latrobe, PA (US)

(21) Appl. No.: 14/651,064

(22) PCT Filed: Sep. 24, 2013
(86) PCT No.: PCT/IB2013/058790

§ 371 Date: Jun. 10, 2015

Related U.S. Application Data
(60) Provisional application No. 61/736,702, filed on Dec. 13, 2012.

(51) Int. Cl. A61M 16/06; B29C 65/70

(52) U.S. Cl. CPC A61M 16/0622; A61M 16/0683; B29C 65/70; A61M 2205/0216; A61M 2207/00; A61M 2210/0618; B29K 2083/00

(57) ABSTRACT

A patient interface device for delivering a flow of breathing gas to an airway of a patient, comprises two or more headgear straps, a cushion, and a thermoplastic hub positioned in the cushion for connection to an elbow or other fluid connector. The cushion is integrally connected to the headgear straps, and the integrated cushion and strap system is overmolded onto the hub.

### CROSS-REFERENCE TO RELATED APPLICATIONS

This patent application claims the priority benefit under 35 U.S.C. §119(e) of U.S. Provisional Application No. 61/736,702 filed on Dec. 13, 2012, the contents of which are herein incorporated by reference.

### BACKGROUND OF THE INVENTION

1. Field of the Invention

The present invention relates to a patient interface device for transporting a gas to and/or from an airway of a user, and, in particular, to a patient interface device comprising a cushion with integral straps.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), or congestive heart failure.

Non-invasive ventilation and pressure support therapies involve the placement of a patient interface device including a mask component on the face of a patient. The mask component comprises, for example, a nasal mask that covers the patient's nose, a nasal cushion having nasal prongs that are received within the patient's nares, a nasal/oral mask that covers the nose and mouth, or a full face mask that covers the patient's face. The patient interface device interfaces the ventilator or pressure support device with the airway of the patient, so that a flow of breathing gas can be delivered from the pressure/flow generating device to the airway of the patient. It is known to maintain such devices on the face of a wearer by headgear having one or more straps adapted to fit over/around the patient's head.

For such patient interface devices, a key engineering challenge is to balance patient comfort against mask stability. This is particularly true in the case of treatment of OSA, where such patient interface devices are typically worn for an extended period of time. As a patient changes sleeping positions through the course of the night, masks tend to become dislodged, and the seal can be broken. A dislodged mask can be stabilized by increasing strapping force, but increased strapping force tends to reduce patient comfort. This design conflict is further complicated by the widely varying facial geometries that a given mask design needs to accommodate. One area where facial geometries vary a great deal is the angle of the base of the nose (known as the nasolabial angle).

Another area where fit and comfort are often a concern is the bridge of the patient's nose, as many patient interface devices will apply a pressure to this area. If this pressure is not able to be managed effectively, either a poor fit or patient discomfort, or both, will result, thereby limiting the effectiveness of the device.

Patient interface devices for providing pressure support therapy, such as CPAP therapy, are typically made up of a frame, a cushion, headgear straps, an elbow connector coupled to a swivel hub, and tubing coupling the elbow connector to a pressure generating device. The frame is used as a central component to which the swivel hub, straps, and cushion can all be attached. To facilitate ease of assembly and to better assure that the components seal well, many of these components are typically produced from plastic or polymeric material that is harder than the strap material.

The SWIFT™ FX mask (available from ResMed Corp., San Diego, Calif.) is an example of a mask that utilizes a typical mask component structure, with the exception that hard materials are not used to create the frame. While this design offers improved comfort to a patient, this design has a significant disadvantage in that it is more difficult to assemble.

### SUMMARY OF THE INVENTION

Accordingly, it is an object of the invention to provide a patient interface device for providing respiratory therapy to a patient that overcomes the shortcomings of conventional patient interface devices. This object is achieved according to one embodiment of the present invention by providing a patient interface device where the cushion and headgear straps are overmolded as one piece onto a hub to connect to an elbow or other fluid connector. This eliminates the need for a frame and reduces an assembly that can comprise up to four components, as perceived by the end user, to only one component.

In addition, silicone around the swivel elbow hub can be designed to act as a shock absorber in that it isolates torque on tubing and an elbow from the features of the mask that seat to a patient's face.

It is an object of this invention to have a face mask without hard material in a frame. Further, it is an object of this invention to incorporate the frame, cushion, swivel hub, elbow, and straps into one continuous and sealed component.

According to the invention, an integrated cushion and headgear strap system for a patient interface device is adapted to provide a regimen of respiratory therapy to a patient. The system comprises two or more headgear straps made of a flexible polymeric material, a cushion made of the flexible polymeric material, and a hub made of a thermoplastic material and positioned in the cushion, the hub being structured to be connected to a fluid connector. Each headgear strap has a proximal end and a distal end, wherein each distal end of a headgear strap is capable of attaching to or engaging a headgear system. The cushion is integrally connected to each proximal end of the headgear straps, and the headgear straps and the cushion form a continuous piece that is overmolded or otherwise attached to the hub.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic front elevational view of an integrated cushion and headgear strap system for a patient interface device adapted to provide a regimen of respiratory therapy to a patient according to one exemplary embodiment of the present invention;

FIG. 2 is a schematic front elevational view of an integrated cushion and headgear strap system for a patient interface device adapted to provide a regimen of respiratory therapy to a patient according to another exemplary embodiment of the present invention;

FIG. 3 is a schematic lateral cross-sectional view of the integrated cushion and headgear strap system of either FIG. 1 or FIG. 2;

FIG. 4 is a schematic front elevational view of an integrated cushion and headgear strap system for a patient interface device adapted to provide a regimen of respiratory therapy to a patient according to another exemplary embodiment of the present invention; and

FIG. 5 is a schematic lateral cross-sectional view of the integrated cushion and headgear strap of FIG. 4.

### DETAILED DESCRIPTION OF THE EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As employed herein, the statement that two or more parts or components “engage” one another shall mean that the parts exert a force against one another either directly or through one or more intermediate parts or components. As employed herein, the term “number” shall mean one or an integer greater than one (i.e., a plurality). As employed herein, durometer hardness shall refer to Shore hardness as measured by a durometer.

In one embodiment of the invention, a patient interface device for delivering a flow of breathing gas to an airway of a patient is provided that includes a mask that optimizes the material properties of flexible polymeric materials to have enough pull force for a cushion on a mask to seal to the patient under a range of pressures, in combination with a thermoplastic swivel airway elbow hub. The thicknesses of the flexible polymeric materials can be varied over the design profile to optimize the pull direction and pull force. Materials of varying elasticity or durometer hardness will be used to vary the stretch and optimize the design of the straps and mask, and the thermoplastic materials will be chosen for suitable stiffness. The different materials will be joined by, for example, overmolding, suitable adhesives, or chemical, mechanical, or thermal bonding.

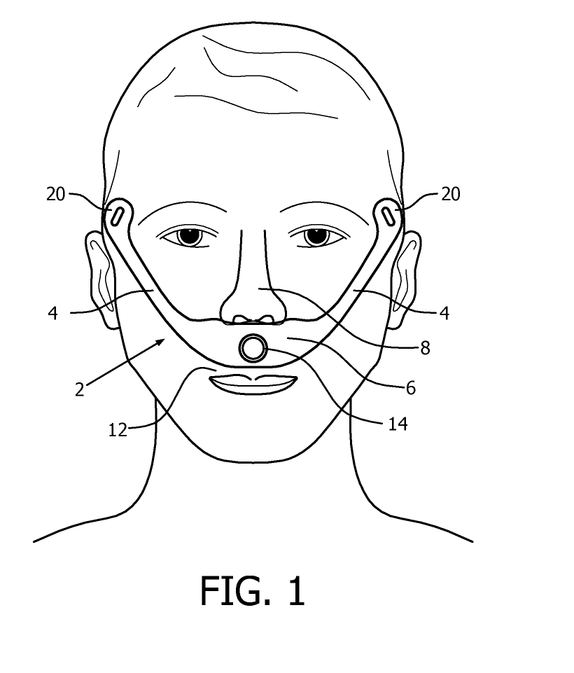

FIG. 1 is a schematic front elevational view of an integrated cushion and headgear strap system for a patient interface device adapted to provide a regimen of respiratory therapy to a patient according to one exemplary embodiment of the present invention. A cushion and headgear strap system 2 comprises headgear straps 4 and a cradle cushion 6, which cradle cushion 6 is to be positioned between a patient's nose 8 and upper lip 12. Straps 4 and cradle cushion 6 are overmolded as one continuous piece onto a circular or spherical hub 14. Hub 14 is shaped and sized to receive and connect to an elbow or other fluid connector (not shown) for coupling cradle cushion 6 to a pressure generating device (not shown). The materials suitable for use in making straps 4, cradle cushion 6, and hub 14 are described in detail elsewhere herein. Straps 4 each have a distal end 20 that is configured to attach to or engage a headgear strap system (not shown).

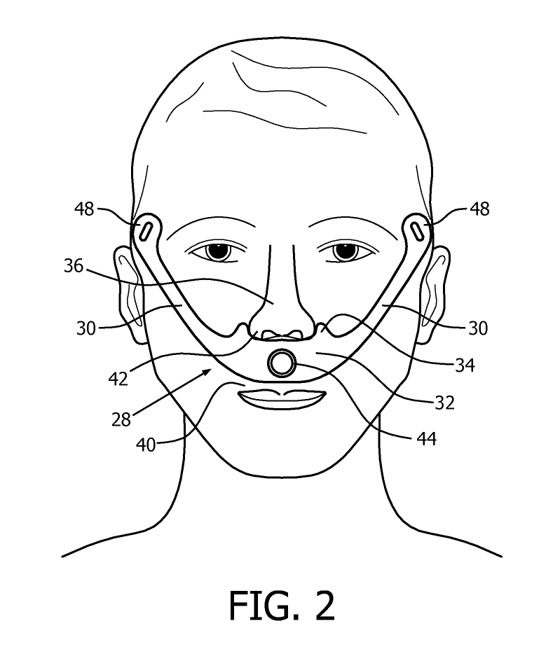

FIG. 2 is a schematic front elevational view of an integrated cushion and headgear strap system for a patient interface device adapted to provide a regimen of respiratory therapy to a patient according to another exemplary embodiment of the present invention. A cushion and headgear strap system 28 comprises headgear straps 30 and a saddle cushion 32, which saddle cushion 32 has substantially parallel projections 34. Saddle cushion 32 is to be positioned between a patient's nose 36 and upper lip 40, with projections 34 engaging or being positioned adjacent to nostrils 42. Straps 30 and saddle cushion 32 are overmolded as one continuous piece onto a circular hub 44. Hub 44 is shaped and sized to receive and connect to an elbow or other fluid connector (not shown) for coupling saddle cushion 32 to a pressure generating device (not shown). The materials suitable for use in making straps 30, saddle cushion 32, and hub 44 are described in detail elsewhere herein. Straps 30 each have a distal end 48 that is configured to attach to or engage a head strap system (not shown).

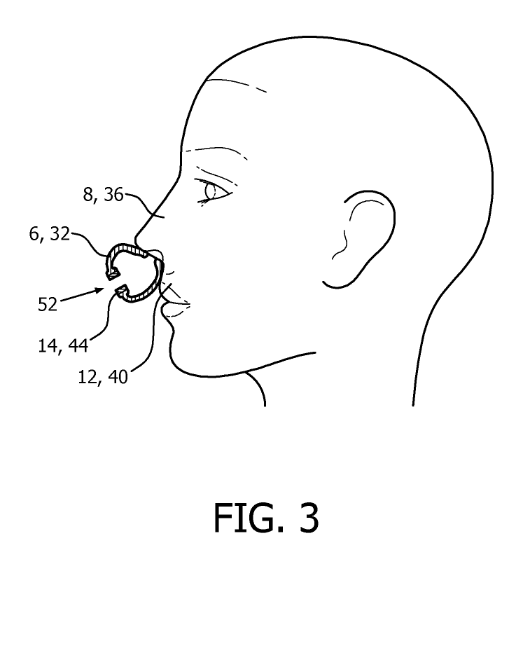

FIG. 3 is a schematic lateral cross-sectional view showing either integrated cushion and headgear strap system 2 of FIG. 1 or integrated cushion and headgear strap system 28 of FIG. 2. As seen in FIG. 3, and as described elsewhere herein, cradle cushion 6 or saddle cushion 32 is positioned in the space below a patient's nose 8, 36 and above the patient's upper lip 12, 40. Cushion 6, 32 has an opening 52 into which hub 14, 44 has been positioned.

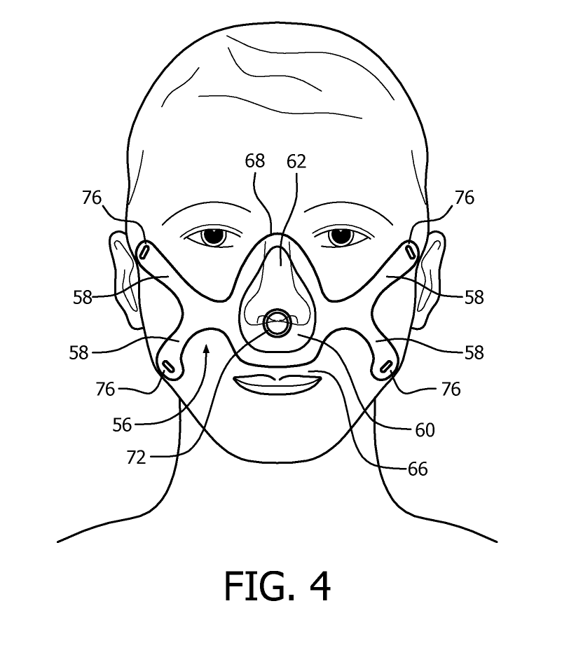

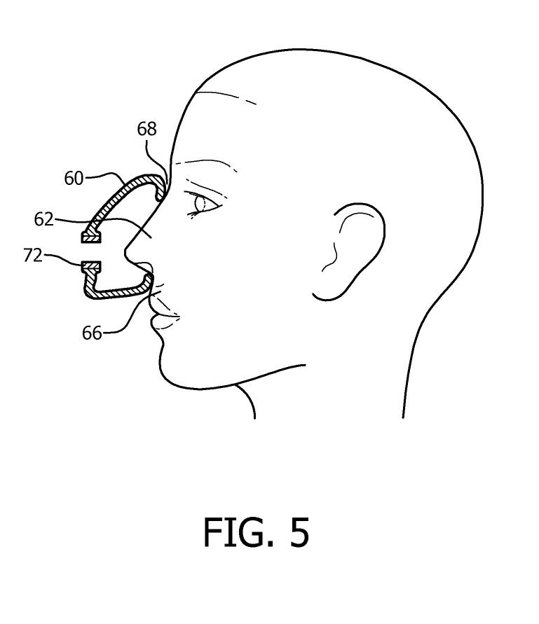

FIG. 4 is a schematic front elevational view and FIG. 5 is a schematic lateral cross-sectional view of an integrated cushion and headgear strap system for a patient interface device adapted to provide a regimen of respiratory therapy to a patient according to another exemplary embodiment of the present invention. A cushion and head strap system 56 comprises four headgear straps 58 and a nasal cushion 60. Nasal cushion 60 fits over a patient's nose 62, extending from about the patient's upper lip 66 to the bridge of a patient's nose, that is, a point 68 almost equal to the level of the patient's eyes 70 or from about 80% to about 100% of the length of the patient's nose 62. Straps 58 and nasal cushion 60 are overmolded as one continuous piece onto a circular hub 72. Hub 72 is shaped and sized to receive and connect to an elbow or other fluid connector (not shown) for coupling nasal cushion 60 to a pressure generating device (not shown). The materials suitable for use in making straps 58, nasal cushion 60, and hub 72 are described in detail elsewhere herein. Straps 58 each have a distal end 76 that is configured to attach to or engage a head strap system (not shown).

Headgear straps 4, 30, 58 and cushions 6, 32, 60 will, in exemplary embodiments, comprise material such as, without limitation, silicone. In the exemplary embodiment, the headgear straps 4, 30, 58 and cushions 6, 32, 60 comprise a material having a durometer hardness of from about 20 Shore A to about 85 Shore A and/or a 100% Modulus of elasticity from about 50 psi to about 1000 psi. On the other hand, hubs 14, 44, 72 each comprise a high durometer, low elasticity material, for example, a polymer or thermoplastic having a durometer hardness of from about 70 Shore A to about 120 Rockwell R and/or a flexural modulus of elasticity from about 500 psi to about 1,000,000 psi.

The thickness of headgear straps 4, 30, 58 and cushions 6, 32, 60 will vary to control stretch positions and compression. A thicker section will require more force to elongate or compress. One skilled in the art would appreciate that variations in thickness and length will permit optimization.

The widths and thicknesses of the cross-sections of headgear straps 4, 30, 58 and cushions 6, 32, 60 can vary, according to desired characteristics and design features. For example, the thicknesses of headgear straps 4, 30, 58 and cushions 6, 32, 60 can be from about 0.5 mm to about 7 mm, and the widths of headgear straps 4, 30, 58 can be from about 7 mm to about 20 mm. The widths of cushions 6, 32, 60 can be from about 6 cm to about 10 cm, whereas the heights of cushions 6, 32, 60 can vary from about 2 cm to about 20 cm.

As described elsewhere herein, in the exemplary embodiment, headgear straps 4, 30, 58 and cushions 6, 32, 60 form a continuous, integrated system that is overmolded onto hub 14, 44, 72. Alternatively, a continuous, integrated system comprising headgear straps 4, 30, 58 and cushions 6, 32, 60 could be attached or bonded to hub 14, 44, 72 by adhesives or in some other mechanical, thermal, or chemical manner.

The preferred polymeric materials useful herein for forming headgear straps 4, 30, 58 and cushions 6, 32, 60 are thermoplastic elastomers, such as polyurethanes, or silicones, that are readily commercially available. Examples of such silicones include Wacker 3003/3009 family of silicones, available from Wacker Chemie AG, Munich, Germany, Bluestar 4310 silicone, available from Bluestar Silicones USA Corp., East Brunswick, N.J., and Shin Etsu 2090 family of silicones, available from Shin Etsu Chemical Co., Ltd., Tokyo.

In the description above it is indicated that certain sections can be overmolded to join such sections together. This process usually includes one material (material X) being molded first into the desired form, and then, once material X has begun to cure from liquid to solid, the next material (material Y) can be molded on top of material X at certain areas, creating cross-linked material and/or chemically bonded materials. As one skilled in the art would appreciate, other methods and techniques for bonding polymeric sections together can be used, including, but not limited to, bonding by chemical, mechanical, or thermal means. Mechanical interlocks can be designed into material X structure (i.e., holes), and when material Y is molded, the uncured material will flow in and around material X structure to create a mechanical interlock. This process is seen on current Philips Respironics mask such as Comfort Gel Full Silicone Flap overmolded to a thermoplastic retaining ring. In instances where materials are chemically bonded, adhesives such as, without limitation, LSR, UV cure adhesive, instant adhesive, RTV, RTV2, can be used to bond material X to material Y.

It can thus be appreciated that the present invention provides an improved patient interface device that is simple to assemble and will comfortably seal to a patient's face.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. An integrated cushion and headgear strap system for a patient interface device adapted to provide a regimen of respiratory therapy to a patient, which comprises:
two or more headgear straps made of a flexible polymeric material, wherein each headgear strap has a proximal end and a distal end, and wherein each distal end of a headgear strap is capable of attaching to or engaging a headgear system,
a cushion made of the flexible polymeric material and integrally connected to each proximal end of the headgear straps, wherein the headgear straps and the cushion form a continuous piece, the cushion having a first side structured to contact a face of the patient responsive to the patient interface device being donned by the patient and a second side opposite the first side, and
a cylindrical hub member have a circular cross section and being made of a thermoplastic material, the hub member being positioned in an opening provided in the second side of the cushion and being surround by the second side of the cushion, the hub member being structured to be connected to a fluid connector.

2. The system of claim 1, wherein the cushion is configured to be positioned between the patient's nose and the patient's upper lip.

3. The system of claim 1, wherein the cushion has two substantially parallel projections that are structured to engage or be positioned adjacent to nostrils of the patient's nose.

4. The system of claim 1, wherein the cushion is configured to extend from the patient's upper lip to a bridge of the patient's nose.

5. The system of claim 4, wherein the two or more headgear straps comprise four headgear straps.

6. The system of claim 1, wherein the headgear straps and the cushion are each comprised of material having a durometer hardness of from about 20 to about 85 shA.

7. The system of claim 1, wherein the hub member is comprised of material having a durometer hardness of from about 70 shA to about 120 Rockwell R.

8. The system according to claim 1, wherein the headgear straps and the cushion are overmolded onto the hub.

9. The system of claim 1, wherein the headgear straps and the cushion are bonded to the hub by adhesives or other chemical, mechanical, or thermal means.

10. A method of making an integrated cushion and headgear strap system for a patient interface device adapted to provide a regimen of respiratory therapy to a patient, comprising:
providing a cylindrical hub member have a circular cross section and being made of a thermoplastic material, the hub member being structured to be connected to a fluid connector; and
coupling a unitary cushion and headgear member to the cylindrical hub member, wherein the cushion and headgear member comprises: two or more headgear straps made of a flexible polymeric material, wherein each headgear strap has a proximal end and a distal end, and wherein each distal end of a headgear strap is capable of attaching to or engaging a headgear system, and a cushion made of the flexible polymeric material and integrally connected to each proximal end of the headgear straps, wherein the headgear straps and the cushion form a continuous piece, the cushion having a first side structured to contact a face of the patient responsive to the patient interface device being donned by the patient and a second side opposite the first side, and wherein the hub member is positioned in an opening provided in the second side of the cushion and is surround by the second side of the cushion.

11. The method of claim 10, wherein the coupling comprises overmolding the cushion and headgear member onto the hub.

12. The method of claim 10, wherein the cushion and headgear member is comprised of material having a durometer hardness of from about 20 to about 85 shA.

13. The method of claim 10, wherein the hub member is comprised of material having a durometer hardness of from about 70 shA to about 120 Rockwell R.
